(19) United States
(12) Patent Application Publication
(10) Pub. No.: US 2017/0231600 A1
(43) Pub. Date: Aug. 17, 2017

(54) CONTINUOUS WAVE DOPPLER DETECTION OF ROSC

(71) Applicant: Physio-Control, Inc., Redmond, WA (US)

(72) Inventor: Daniel W. Piraino, Seattle, WA (US)

(21) Appl. No.: 15/365,687

(22) Filed: Nov. 30, 2016

Related U.S. Application Data
(60) Provisional application No. 62/261,250, filed on Nov. 30, 2015.

(51) Int. Cl. A61B 8/08; A61B 8/02; A61N 1/39

(52) U.S. Cl. CPC A61B 8/488; A61N 1/3925; A61B 8/0883; A61N 1/3987; A61B 8/02

(57) ABSTRACT

An event-driven medical treatment data notification system is disclosed. Embodiments are directed to a treatment protocol for detecting Return of Spontaneous Circulation by employing a Doppler effect analysis on a signal transmitted at a patient's heart. A reflected signal is evaluated to determine if the patient's heart is moving, thereby indicating that the patient's heart is beating and, therefore, that blood is flowing.

### CROSS REFERENCE TO RELATED APPLICATIONS

This patent application claims the benefit of and priority to U.S. Provisional Patent Application Ser. No. 62/261,250 filed on Nov. 30, 2015, entitled “Continuous Wave Doppler to Detect Return of Spontaneous Circulation (ROSC),” the disclosure of which is hereby incorporated by reference for all purposes.

### TECHNICAL FIELD

The disclosed subject matter pertains generally to the area of medical devices, and more specifically to the area of medical devices for the detection of spontaneous circulation.

### BACKGROUND INFORMATION

Many types of acute cardiac syndrome (ACS) present themselves as an absence of spontaneous circulation, meaning that a person's blood has stopped flowing for various reasons. For a patient to enjoy a complete recovery, obtaining the Return of Spontaneous Circulation (ROSC) is very important. Accordingly, it is important to determine if ROSC has occurred in any cardiac arrest treatment plan. Currently ROSC is determined primarily by whether a patient has a pulse. Once the patient has regained a pulse, defibrillation and CPR therapy should be withheld, and the patient should be given basic life support (oxygen, etc.) and monitored in case the patient suffers another cardiac arrest. Existing methods of detecting ROSC, such as palpating a pulse on the patient's wrist, carotid or femoral arteries, are unreliable. Other methods—such as SPO2, CO2 and NIBP—may also be used, but these methods are not available without attaching more equipment and are not available at all to the user of an Automated External Defibrillator (AED).

A superior technique for easily and reliably detecting the occurrence of ROSC has eluded those skilled in the art, until now.

### SUMMARY OF EMBODIMENTS

Embodiments are directed to a method and apparatus for treating a patient experiencing a cardiac incident which causes the patient's heart to stop beating. Certain embodiments are directed to a technique for detecting the Return of Spontaneous Circulation (ROSC) by transmitting a signal in the direction of the patient's heart and analyzing a return signal that may be reflected by the patient's heart. By employing a Doppler analysis, a determination can be made whether the heart appears to be beating. If the heart is beating, an assumption is made that the heart is flowing blood. In another embodiment, a transducer assembly is provided which may be used in a Doppler analysis to determine if a patient's heart is beating. In one particular embodiment, a plurality of transducer elements is provided in the transducer assembly. In addition, in this embodiment a spacing between adjacent transducers is proportional to an estimated intercostal distance. In a further refinement, spacing between two sets of adjacent transducer elements each have a different proportion to an estimated intercostal distance.

### BRIEF DESCRIPTION OF THE DRAWINGS

FIG. 1 is a diagram of a scene where an external defibrillator is used to try and save the life of a person in accordance with an embodiment.

FIG. 2 is a table listing two main types of the external defibrillator shown in FIG. 1, and by whom they might be used.

FIG. 3 is a diagram showing components of an external defibrillator made according to embodiments.

FIG. 4 is a conceptual illustration of a scenario using Doppler techniques to detect ROSC, in accordance with one embodiment.

FIG. 5 is a conceptual illustration of a scenario using Doppler techniques to detect ROSC, in accordance with one embodiment.

FIG. 6 is a conceptual illustration of one implementation of a transducer assembly in accordance with one embodiment.

FIG. 7 is a procedural flow diagram generally illustrating a typical cardiac arrest algorithm that may be performed on an individual suffering from a cardiac event.

FIG. 8 is a procedural flow diagram generally illustrating one embodiment of a process performed by an illustrative system for registering recipients to receive treatment data based on events for which that recipient is interested.

### DETAILED DESCRIPTION

Generally described, the disclosure is directed at the detection of the return of spontaneous circulation (ROSC) by detecting whether a patient's heart is beating using Doppler techniques. For ROSC detection purposes, it is not necessarily important to know if blood is flowing to know whether a patient's heart is beating. The heart walls move at a speed easily detected by Doppler ultrasound and at an amplitude some 1000 times higher than blood flow. In addition, a beating heart moves in multiple directions. Accordingly, the disclosed embodiments determine the occurrence of ROSC by applying Doppler techniques to determine if the heart is moving rather than if blood is flowing.

This disclosure begins with a description of one example of a medical device that may be used in specific embodiments. Next is a discussion of one specific example of a treatment for detecting ROSC using Doppler principles. Finally, one embodiment of an illustrative transducer assembly in accordance with a preferred embodiment is shown and described.

### Description of Operative Environment for Embodiments

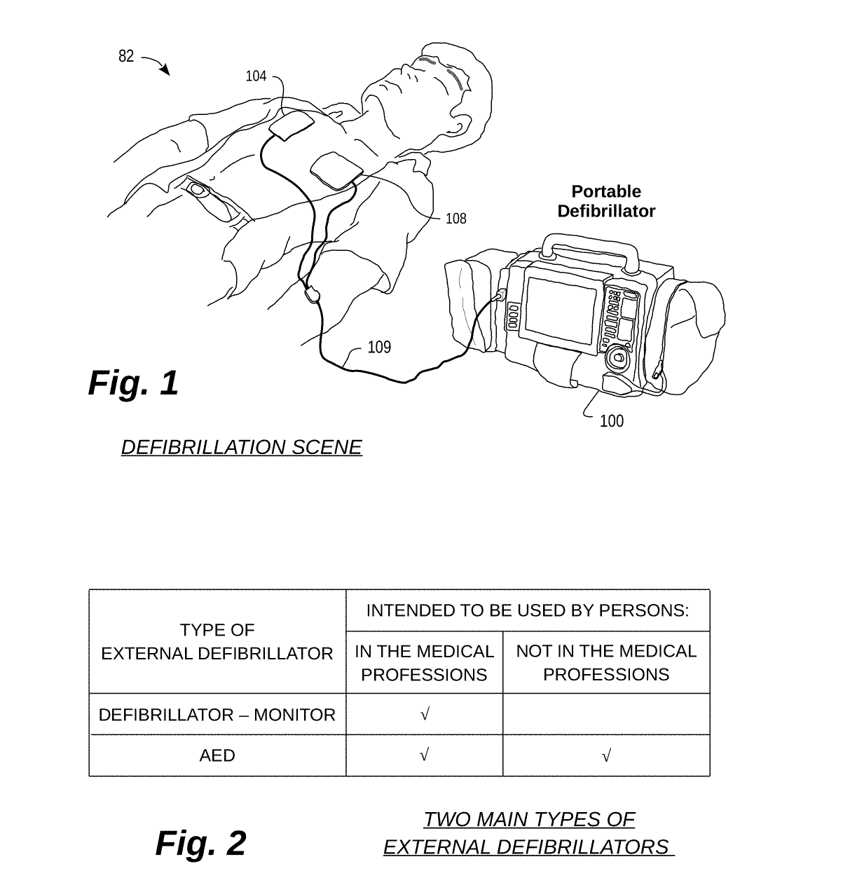

FIG. 1 is a diagram of a defibrillation scene. A person 82 is lying supine. Person 82 could be a patient in a hospital or someone found unconscious. Person 82 is experiencing a medical emergency, which could be, by way of an example, Ventricular Fibrillation (VF).

A portable external defibrillator 100 has been brought close to person 82. The portable external defibrillator can also be a hybrid monitor/defibrillator 82. At least two defibrillation electrodes 104, 108 are usually provided with external defibrillator 100. Electrodes 104, 108 are coupled with external defibrillator 100 via electrode leads 109. A rescuer (not shown) has attached electrodes 104, 108 to the skin of person 82. Defibrillator 100 is monitoring cardiac rhythms and potentially administering, via electrodes 104, 108, a brief, strong electric pulse through the body of person 82. The pulse, also known as a defibrillation shock, goes through the person's heart in an attempt to restart it, for saving the life of person 82.

Defibrillator 100 can be one of different types, each with different sets of features and capabilities. The set of capabilities of defibrillator 100 is determined by planning who would use it, and what training they would be likely to have. Examples are now described.

FIG. 2 is a table listing examples of types of external defibrillators and their primary intended users. A first type of defibrillator 100 is generally called a defibrillator-monitor (or monitor-defibrillator) because it is typically formed as a single unit in combination with a patient monitor. Alternatively, the defibrillator-monitor may be a modular device with separable components. For example, in one alternative embodiment, the defibrillator-monitor may include a base component and a plurality of detachable pods or modules. Each pod communicates with the base component, perhaps wirelessly. Certain pods may be used to collect information about a patient, such as vital statistics. One example of such an alternative system is described in U.S. Pat. No. 8,738,128 entitled “Defibrillator/Monitor System Having A Pod With Leads Capable Of Wirelessly Communicating,” the disclosure of which is hereby incorporated by reference for all purposes. A defibrillator-monitor is intended to be used by medical professionals, such as doctors, nurses, paramedics, emergency medical technicians, etc. Such a defibrillator-monitor is generally intended to be used in a pre-hospital or hospital scenario.

As a defibrillator, the device can be one of different varieties, or even versatile enough to be able to switch among different modes that individually correspond to the varieties. One variety is that of an automated external defibrillator (AED), which can determine whether a shock is needed and, if so, charge to a predetermined energy level and instruct the user to administer the shock. Another variety is that of a manual defibrillator, where the user determines the need and controls administering the shock.

As a patient monitor, the device has features additional to what is minimally needed for mere operation as a defibrillator. These features can be for monitoring physiological indicators of a person in an emergency scenario. These physiological indicators are typically monitored as signals, such as a person's full ECG (electrocardiogram) signals, or impedance between two electrodes. Additionally, these signals can be about the person's temperature, non-invasive blood pressure (NIBP), arterial oxygen saturation/pulse oximetry (SpO2), the concentration or partial pressure of carbon dioxide in the respiratory gases, which is also known as capnography, and so on. These signals can be further stored and/or transmitted as patient data. In some embodiments, a patient monitor device does not include defibrillation functions and may optionally include other functions such as, for example, blood marker testing functionality, ventilation functionality, airway management functionality, CPR feedback functionality, and/or chest compression functionality.

A second type of external defibrillator 100 is generally called an AED, An AED typically makes the shock/no shock determination by itself, automatically. It can typically sense enough physiological conditions of the person 82 using only the defibrillation electrodes 104, 108 shown in FIG. 1. An AED can either administer the shock automatically, or instruct the user to do so, e.g. by pushing a button.

There are other types of external defibrillators in addition to those listed in FIG. 2. For example, a hybrid defibrillator can have aspects of an AED and also of a defibrillator-monitor. A usual such aspect is additional ECG monitoring capability.

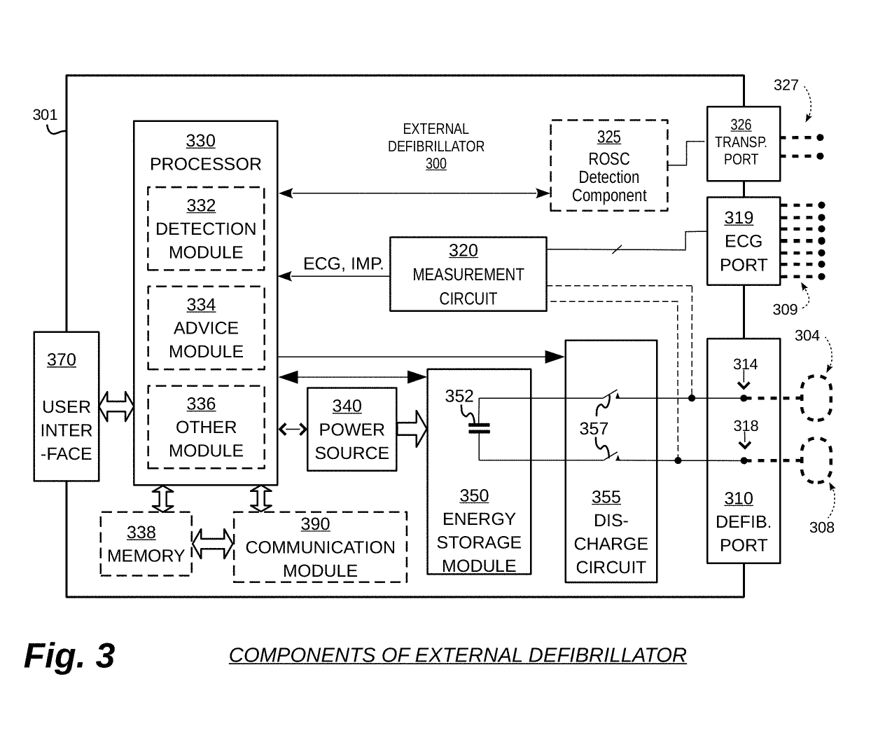

FIG. 3 is a diagram showing components of an external defibrillator 300 made according to embodiments. These components can be, for example, in external defibrillator 100 of FIG. 1. The components shown in FIG. 3 can be provided in a housing 301, also known as a casing. It will be appreciated that, in other embodiments, these components may be implemented in separate housings or as sub-components of various other devices.

External defibrillator 300 is intended for use by a user, who is frequently the rescuer. Defibrillator 300 typically includes a defibrillation port 310, such as a socket in housing 301. Defibrillation port 310 includes nodes 314, 318. Defibrillation electrodes 304, 308, which can be similar to electrodes 104, 108, can be plugged in defibrillation port 310, so as to make electrical contact with nodes 314, 318, respectively. It is also possible that electrodes can be connected continuously to defibrillation port 310, etc. Either way, defibrillation port 310 can be used for guiding an electrical charge to person 82 via electrodes 304, 308. The electrical charge may be stored in defibrillator 300, as discussed below.

If defibrillator 300 is a defibrillator-monitor, as was described with reference to FIG. 2, it will frequently also have an ECG port 319 in housing 301, for plugging in ECG lead wires 309. ECG lead wires 309 can sense an ECG signal, such as any of the ECG lead signals that comprise a common 12-lead ECG recording. Other types of ECG lead signals are equally applicable. A defibrillator-monitor could have additional ports that are not shown.

Defibrillator 300 may also include a measurement circuit 320, which receives physiological signals from ECG port 319, and also from other ports, if provided. These physiological signals are sensed, and information about them is rendered by measurement circuit 320 as data, or other signals, etc.

Defibrillator 300 also includes a processor 330, which may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 330 can be considered to have a number of modules. One such module can be a detection module 332, which senses outputs of measurement circuit 320. Detection module 332 can include a VF detector. Thus, the person's sensed ECG can be used to determine whether the person is experiencing VF.

Another such module in processor 330 can be an advice module 334, which arrives at advice based on outputs of detection module 332. The advice can be to shock, to not shock, to administer other forms of therapy, and so on. If the advice is to shock, the AED will automatically charge the energy storage module 350. Some external defibrillator embodiments merely report the shock advice to the user, and prompt them to do it. Other embodiments further execute the advice, by administering the shock automatically. If the advice is to administer CPR, defibrillator 300 may further issue prompts for it, and so on. Processor 330 can include additional modules, such as other module 336, for other functions too numerous to list here.

Defibrillator 300 optionally further includes a memory 338, which can work together with processor 330. Memory 338 may be implemented in any number of ways. Such ways include, by way of example and not of limitation, nonvolatile memories (NVM), read-only memories (ROM), random access memories (RAM), any combination of these, and so on. Memory 338, if provided, can include programs for processor 330, and so on. In addition, memory 338 can store prompts for the user, etc. Moreover, memory 338 can store patient data, such as, for example, data regarding how much fluid may have been administered to patient 82 as detected by the flow monitor component 325.

In one embodiment, the defibrillator 300 includes a ROSC detection component 325. In one specific implementation, the ROSC detection component 325 includes functions which, when executed by processor 330, operate to detect the occurrence of ROSC through the use of a sonar wave analysis. In one particular embodiment, the sonar wave analysis implements any appropriate Doppler detection technique, such as autocorrelation or Fourier transform processing, to determine Doppler characteristics of an object. The ROSC detection component may be coupled to a transducer via a transducer port 326, which causes a sonar wave to be generated and transmitted, via a transducer, to a patient. A return signal received at the transducer may be communicated back to the ROSC detection component via the transducer port 326 using transducer wires 327. Alternatively, the transducer port 326 may be integrated into another element of the defibrillator 300, such as the ECG port 319 or the defibrillation port 310.

For the purpose of determination or ROSC it is sufficient to determine if the heart wall is moving in a rhythmic manner. Existing Doppler ultrasound for cardiac purposes remove the echoes from the heart walls by using a low pass filter called a Wall Filter. However, the disclosed embodiments depart from conventional teachings and eliminate or reduce the Wall Filters because that represents the desired signal and not the artifact for this purpose.

ROSC detection component 325 may provide notice of its analysis in many ways. In one example, the ROSC detection component 325 may be an automatic detector which provides an on-screen indication, via user interface 370, of its analysis. Alternatively, ROSC detection component 325 may output to a more direct indicator, such as a speaker which is commonly used in Doppler devices, In still other embodiments, ROSC detection component 325 may be implemented to provide output using other intermediate detectors, such as level-detectors, average frequency, etc.

Defibrillator 300 typically also includes a power source 340. To enable portability of defibrillator 300, power source 340 typically includes a battery. Such a battery is typically implemented as a battery pack, which can be rechargeable or not. Sometimes, a combination of rechargeable and non-rechargeable battery packs is used. Other embodiments of power source 340 can include AC power override, for where AC power will be available, and so on. In some embodiments, power source 340 is controlled by processor 330.

Defibrillator 300 additionally includes an energy storage module 350. Module 350 is where some electrical energy is stored, when preparing it for sudden discharge to administer a shock. Module 350 can be charged from power source 340 to the right amount of energy, as controlled by processor 330. In typical implementations, module 350 includes one or more capacitors 352, or the like.

Defibrillator 300 moreover includes a discharge circuit 355. Discharge circuit 355 can be controlled to permit some or all of the energy stored in module 350 to be discharged to nodes 314, 318, and thus also to defibrillation electrodes 304, 308. Discharge circuit 355 can include one or more relays or switches 357. Those can be made in a number of ways, such as by an H-bridge, or the like.

Defibrillator 300 further includes a user interface 370. User interface 370 can be made in any number of ways. For example, interface 370 may include a screen, to display what is detected and measured, provide visual feedback to a rescuer for their resuscitation attempts, and so on. User interface 370 may also include a speaker to issue audible signals, such as voice prompts, or the like. The user interface 370 may issue prompts to the user, visually or audibly, so that the user can administer CPR, for example. Interface 370 may additionally include various controls, such as pushbuttons, keyboards, touch screens, and so on. In addition, discharge circuit 355 can be controlled by processor 330, or directly by user via user interface 370, and so on.

Defibrillator 300 can optionally include other components. For example, a communication module 390 may be provided for communicating with other machines. Such communication can be performed wirelessly, or via wire, or by infrared communication, and so on. This way, data can be communicated, such as patient data, incident information, therapy attempted, CPR performance, and so on.

Treatment for Detection of ROSC with Doppler

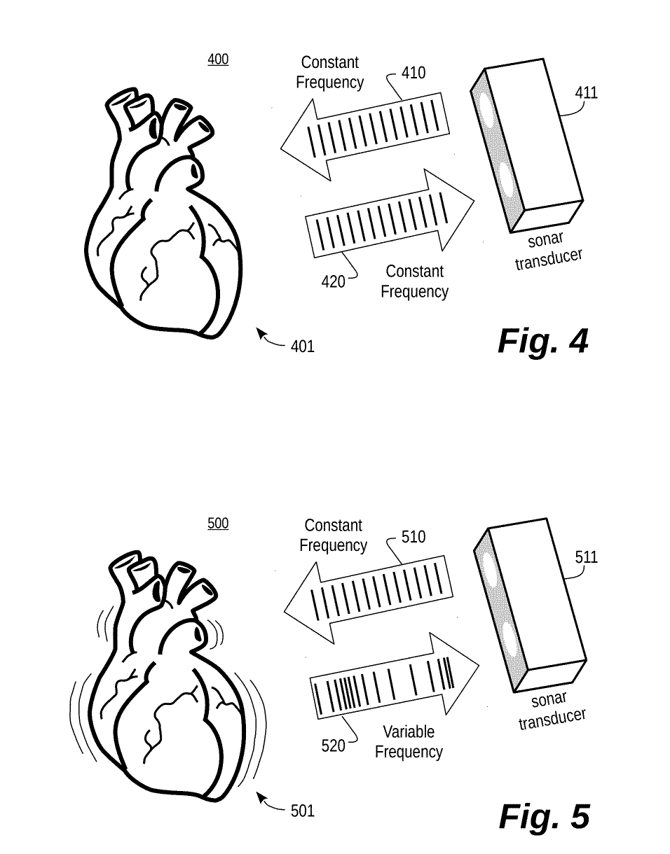

FIGS. 4 and 5 are conceptual illustrations showing the operation of one embodiment. As is known in the art, if a continuous wave (e.g., a sound wave or electromagnetic wave) is transmitted at a constant frequency 410 and impinges on an object that is still with respect to the wave transmitter (e.g., transducer 411), the return wave reflected from that object will also have the same constant frequency 420. Accordingly, as shown in FIG. 4, a transducer 411 transmits a continuous sound wave at a constant frequency 410. That sound wave is transmitted generally in the direction of a patient's heart 401. In the case where the heart 401 is still, meaning that it is not beating, the transmitted sound wave 410 is reflected back to the transducer 411 as essentially the same continuous sound wave 420 with no significant alteration to the frequency of the wave. Thus, in accordance with the teachings of this disclosure, the reflected wave 420 is analyzed to determine if it is returning at essentially the same, unaltered frequency as the transmitted wave 410 which indicates that the heart is not moving, which in turn indicates the heart is not beating. Since the heart 401 is not beating, it is not pumping blood and there is no ROSC.

Referring now to FIG. 5, another scenario is shown in which the patient's heart 501 is beating. As shown in FIG. 5, the transducer 511 transmits a continuous wave 510 at a constant frequency. However, unlike the scenario shown in FIG. 4, the continuous wave 510 impinges upon a beating heart 501. As is known, a beating heart moves in several directions. The beating heart 501 constricts and expands in a pumping motion. In addition, the beating heart 501 may also undergo substantially lateral movement within the chest cavity. Accordingly, those movements alter the transmitted continuous wave 510 such that a detectable frequency difference occurs. The reflected wave 520 is returned as a substantially variable frequency wave. Such frequency alteration is created by the Doppler effect. The variable frequency will be rhythmic with the beating heart. In other words, conceptually there will be 60 changes in the detected frequency per minute if the patient has a pulse of 60 beats per minute.

Accordingly, embodiments may monitor the variations in the frequency of the reflected wave 520 to identify or estimate the heart rate. As mentioned, a variable frequency reflected wave 520 that indicates a rhythmic heart beat in the range of 60 beats per second suggests the presence of a normal heartbeat (a ROSC condition). Alternatively, a variable frequency that changes at a rate in excess of 60 transitions per minute suggests the presence of an abnormally-high heart rate or perhaps Ventricular Tachycardia (“VT”). An excessively high rate at which the variable frequency changes, or perhaps a drastically-irregular change of the variable frequency, may suggest Ventricular Fibrillation (“VF”), which is an extremely dangerous heart condition. Accordingly, in addition to monitoring for the presence of a variable frequency reflected wave, embodiments may be implemented that closely monitor the rate of change of the variable frequency reflected wave to provide even further information about the state of the patient's heart rate.

### Illustrative Transducer Assembly

It should be noted that ultrasound waves do not travel easily through bone. Sound waves should be transmitted, if at all possible, through soft tissue. Accordingly, the transducer used in a preferred embodiment should ideally be placed against the patient at a location which has no bone between the transducer and the patient's heart. One likely location that would prove acceptable is along the patient's left side with the transducer located in the intercostal space between two of the patient's ribs. However, such a location on the patient may be difficult to identify by a caregiver under the stress of life-saving treatment or inadequate training. Accordingly, one embodiment of a transducer assembly is implemented to help ameliorate the necessity for an accurate placement of the transducer against the patient.

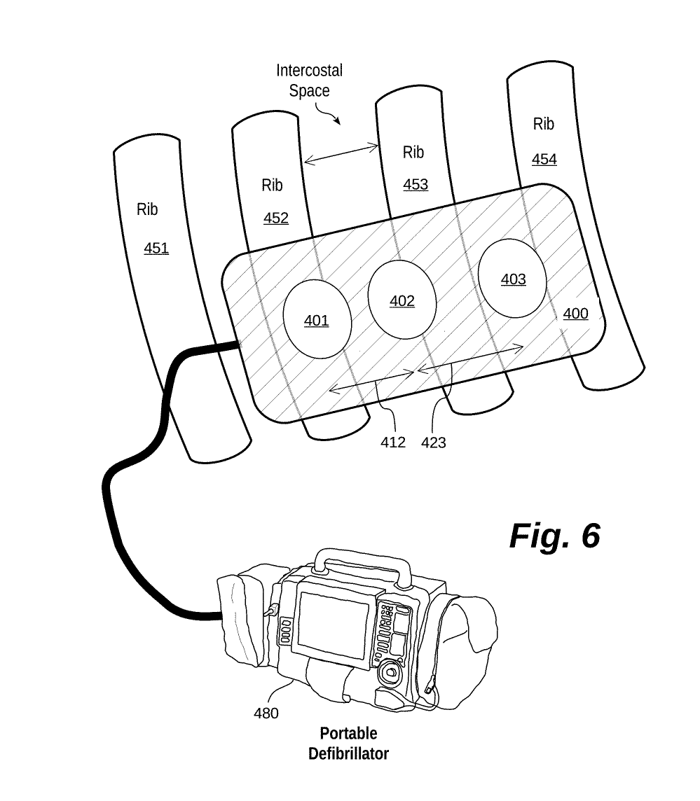

FIG. 6 is a conceptual illustration of a transducer assembly 400 that implements one preferred embodiment. The transducer assembly 400 of this embodiment includes more than a single transducer. In this particular implementation, the preferred transducer assembly 400 includes three transducers (401, 402, 403). Although three transducers are disclosed in one preferred embodiment, it will be appreciated that any number of transducers from one to many may be used in alternative implementations. The disclosed embodiment makes use of three for illustrative purposes only.

In this particular embodiment, the three transducers (401, 402, 403) are separated on the sonar paddle 400 at either the same or varying spacings. In one preferred embodiment, the spacing between one transducer and its adjacent transducer (e.g., transducer 401 and transducer 402) may be some distance that is substantially different than some pre-determined distance, such as the average distance between two ribs on an average-sized human being.

For example, if a typical intercostal distance is X, the distance between two adjacent transducers may be ½ of X. Such a spacing helps to reduce the likelihood that two adjacent transducers are both obstructed by the patient's rib (e.g., rib 452 and rib 453). In one particular enhancement, a third transducer (e.g, transducer 403) may also be included which has a spacing between its nearest neighbor that is different than the spacing between other transducers on the transducer assembly 400. In one example, there is a different spacing 423 between transducer 402 and transducer 403 than the spacing 412 between transducer 401 and transducer 402. Again, if the intercostal distance is X, the distance between a second pair of transducers may be ⅓ or ¼ of X. In this way, the likelihood that all three transducers are obstructed by a rib is reduced even further.

In operation, a software component—such as the ROSC detection component 325 shown in FIG. 3—may be used to programmatically identify the particular transducer that is experiencing a good return signal. In other words, the software component may analyze return signals measured by one or more of the transducers (401, 401, 403) on the transducer assembly 400 and determine which one or more is experiencing the highest quality signal. Alternatively, the software component may compute an average or other weighted quantitative value based on two or more of the transducers which are experiencing a reflected signal, or as another alternative combine them.

In this particular embodiment, the transducer assembly 400 is illustrated as a stand-alone component. However, in other embodiments the transducer assembly 400 may be combined with or integrated into another component. In one example, a portable external defibrillator 480 may be specially configured to support the Doppler detection of ROSC. In such an embodiment, the transducer assembly 400 may be combined with or integrated into a set of ECG leads, one or more defibrillation electrodes, or some other component of the portable defibrillator 480. In this way, the function of detecting ROSC may be incorporated into a medical device which is already in use in medical emergency situations, thereby eliminating a need to employ yet another, separate medical device.

The disclosed embodiments represent an improvement to other systems because the disclosed embodiments eliminate a number of shortcomings present with other systems. For example, using other methods—those that seek to detect blood flow—a special ultrasound transducer is placed either near the apex of the heart or in the supersternal notch and then manipulated until it is pointed into the right or left ventricular outflow tracts. The placement of the transducer is important for those other approaches because to detect blood flow the transducer must be placed as much in parallel to the direction of flow as possible because the Doppler frequency is reduced by a factor of the cosine of the angle between the ultrasound beam and the target flow. However, in accordance with the disclosed embodiments, the transducer assembly 400 may be directed perpendicular to the patient's chest wall at a place in which the beam would be more likely to intersect with some part of the heart wall, such as in the 3rd or 4th intercostal space on the patient's left side. In addition the heart wall reflects much more of the ultrasonic energy than blood making detection easier. The disclosed embodiment reduces the skill that may be necessary on the part of the operator using the transducer assembly, which results in fewer mistakes made during critical treatment.

### Illustrative Process For Implementing Embodiments

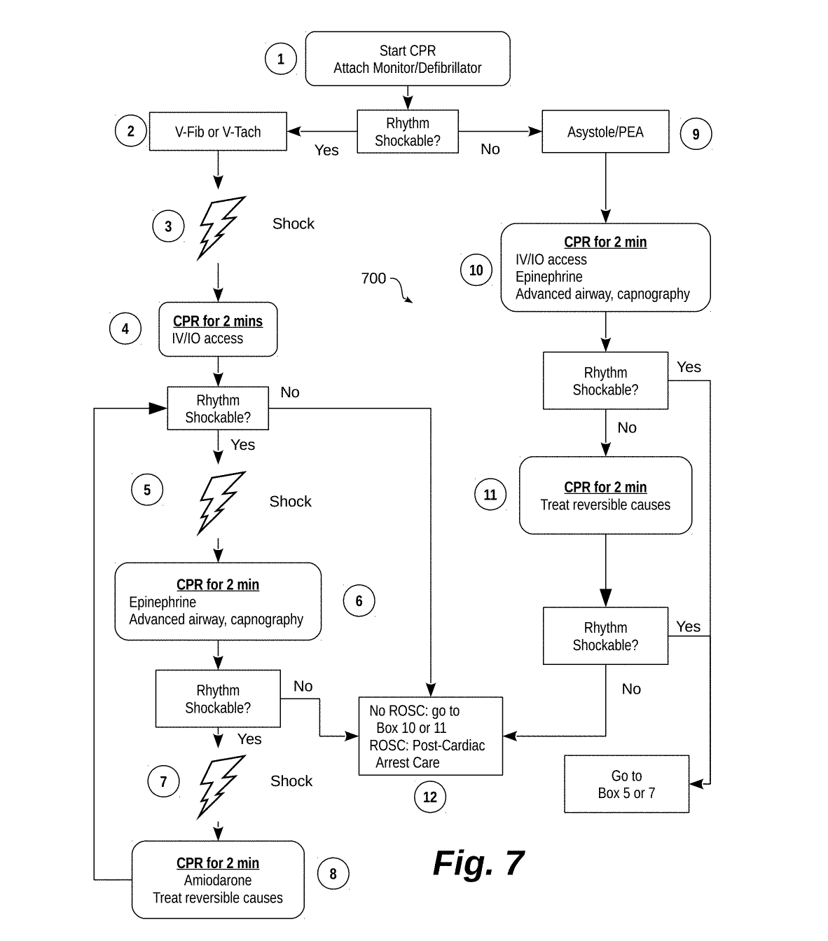

Implementations of the disclosed embodiments may be used during performance of cardiac care on a patient. Preferred cardiac arrest treatment protocols (also known as cardiac arrest algorithms) have been published by leading health care providers in the industry, such as the American Heart Association. FIG. 7 is a procedural flow diagram generally illustrating one such cardiac arrest algorithm. Those skilled in the art will appreciate that the cardiac arrest algorithm illustrated in FIG. 7 is used, generally, by medical professionals and para-professionals in the treatment of patients in physical distress where a cardiac arrest is suspected. Generally stated, the cardiac arrest algorithm 700 provides that the treating individual evaluates the patient's ECG, delivers defibrillation shocks, and performs CPR in cycles until the patient's ECG is in a nonshockable rhythm. Then CPR is performed until ROSC is detected, at which point the unnecessary treatment terminates. All the while, the ECG would be continuously monitored to see if it has gone back into a shockable rhythm.

It will be appreciated that a significant aspect of the standard cardiac arrest algorithm involves detecting ROSC in the patient. In accordance with disclosed embodiments, the standard cardiac arrest algorithm illustrated in FIG. 7 may be modified, supplemented, or perhaps overlaid with one or more implementations of the disclosed embodiments to facilitate the detection of ROSC during treatment. In various embodiments, a defibrillator in use to deliver shocks to the patient may employ an implementation of the disclosure. Alternatively, a separate ROSC detection device may be used in conjunction with a defibrillator or monitor. In this way, the patient may be monitored for ROSC without necessarily deviating from the cardiac arrest algorithm, thereby enhancing the patient's treatment and, concomitantly, the patient's potential for survival. What follows is

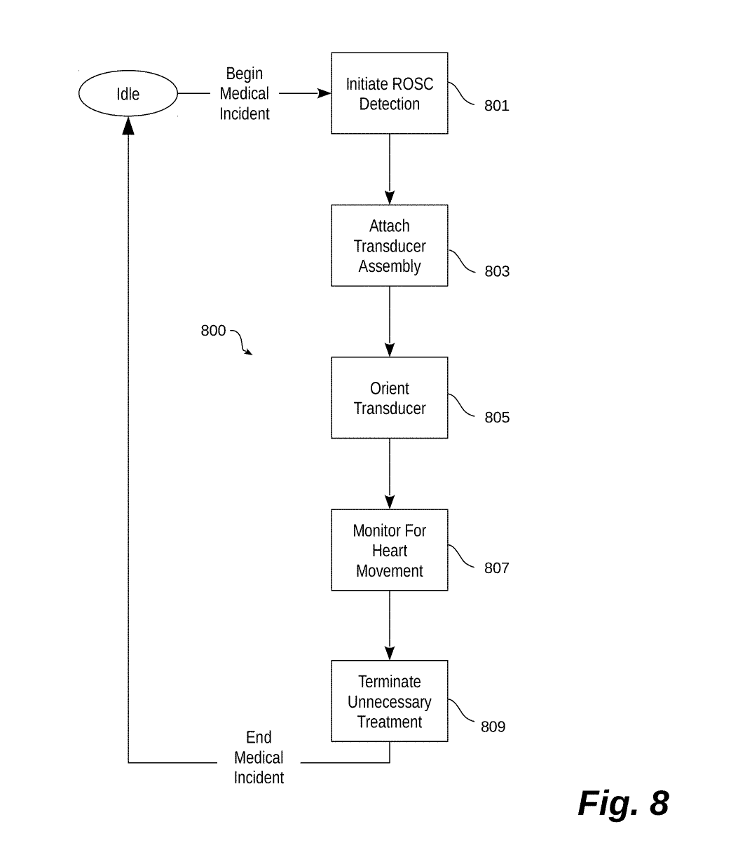

FIG. 8 is a functional flow diagram generally illustrating a treatment protocol 800 for the detection of ROSC using Doppler techniques, in accordance with one particular embodiment. The illustrative treatment protocol 800 may be performed in conjunction with or as a component of the cardiac arrest algorithm just described. For example, this protocol may be performed in addition to or conjunction with other treatment protocols, such as monitoring the patient's vital signs, ECG, etc. and performing defibrillation, cardioversion, and/or CPR. The treatment protocol 800 begins when a medical incident has occurred which presents itself as a cardiac emergency. In accordance with this embodiment, the protocol describes, generally, steps performed by a treating individual (e.g., a medical professional) providing cardiac care to a patient experiencing cardiac arrest.

To begin, a medical incident occurs that presents itself as a form of acute cardiac syndrome, such as a heart attack, in which it becomes preferred to determine whether the patient has spontaneous circulation. In such a case, the treating individual begins (step 801) by initiating the ROSC detection technique of one particular embodiment. In this example, the ROSC detection technique involves the detection of ROSC based on a Doppler sonar evaluation of the patient. Initiating the protocol may take any one of many forms, but generally requires only that the treating individual determine that detection of ROSC should occur, and may additionally include activating a Doppler detection system, such as may be included in a defibrillator, a defibrillator/monitor, or any other medical device configured with Doppler detection of ROSC.

The treating individual also attaches a transducer assembly to the patient (step 803). In this particular embodiment, the transducer assembly is configured in accordance with the embodiment illustrated in FIG. 6 and described above. Accordingly, the transducer assembly includes a plurality of transducers with at least two spaced apart a distance that is selected to improve the likelihood that at least one transducer is located substantially in the intercostal space between two of the patient's ribs. In other embodiments, a transducer assembly may be employed that does not implement a spacing between transducer elements to improve the likelihood of avoiding a rib. For example, an embodiment for use in an environment where only skilled technicians will be using the device, less forgiving transducer assemblies may be implemented.

In this embodiment, the treating individual orients the transducer assembly generally toward the patient's heart (step 805). As should be apparent from the foregoing teachings, the disclosed embodiments eliminate any need to orient the transducer assembly in any particular angle with respect to one of the heart's inflow or outflow tracts, an artery or a vein. Rather, the disclosed embodiments rely on the assumption that if the heart is beating rhythmically, blood is probably flowing. Accordingly, the transducer assembly need only be oriented such that a detection pulse (such as a continuous wave sonar pulse) be transmitted substantially directly toward any part of the patient's heart.

With the transducer assembly attached and oriented, this embodiment monitors for heart movement (step 807). In the disclosed embodiments, monitoring for heart movement is performed by transmitting a substantially continuous wave sonar (e.g., ultrasound) pulse in the direction of the heart and analyzing the return signal. The analysis may employ any appropriate Doppler analysis to determine if the heart is moving. In one embodiment, the Doppler analysis is performed by using a software agent, such as the ROSC detection component 325 shown in FIG. 3 and described above.

As noted above, it may be detrimental to the patient's recovery if CPR or other treatment continues after the patient obtains ROSC. Accordingly, if ROSC is detected, the caregiver should cease performing any treatment that would be counterproductive to the patient's recovery, such as CPR. More specifically, once it has been determined that ROSC has likely occurred because the heart is beating, any treatment should cease that is directed at either attempting to restart the heart beating or to perform heart compressions (step 809).

In summary, the disclosed embodiments overcome shortcomings of existing systems by obviating the need to either manually attempt to detect a pulse (which can be unreliable) and eliminating any need to align an ultrasound transducer with a vein or artery of the patient's heart. In these and other ways, which will become apparent upon a study of the disclosed teachings, these embodiments provide a superior treatment technique and transducer assembly for the detection of ROSC in a patient experiencing a cardiac incident.

Other embodiments may include combinations and sub-combinations of features described above or shown in the Figures, including, for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

In the foregoing description, numerous details have been set forth in order to provide a sufficient understanding of the described embodiments. In other instances, well-known features have been omitted or simplified to not unnecessarily obscure the description.

A person skilled in the art in view of this description will be able to practice the disclosed invention. The specific embodiments disclosed and illustrated herein are not to be considered in a limiting sense. Indeed, it should be readily apparent to those skilled in the art that what is described herein may be modified in numerous ways. Such ways can include equivalents to what is described herein. In addition, the invention may be practiced in combination with other systems. In general, the various features and processes described above may be used independently of one another, or may be combined in different ways. For example, this disclosure includes other combinations and sub-combinations equivalent to: extracting an individual feature from one embodiment and inserting such feature into another embodiment; removing one or more features from an embodiment; or both removing a feature from an embodiment and adding a feature extracted from another embodiment, while providing the advantages of the features incorporated in such combinations and sub-combinations irrespective of other features in relation to which it is described. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example examples. The example systems and components described herein may be configured differently than described. For example, elements may be added to, removed from, or rearranged compared to the disclosed example examples. The following claims define certain combinations and subcombinations of elements, features, steps, and/or functions, which are regarded as novel and non-obvious. Additional claims for other combinations and subcombinations may be presented in this or a related document.

What is claimed is:

1. A medical device, comprising:
a transducer assembly to detect a cardiac condition in a patient, the transducer assembly comprising a plurality of transducer elements, each transducer element in the transducer assembly being configured to transmit a sonic pulse and to receive a return signal, the return signal being at least a partial reflection of the transmitted sonic pulse, at least one pair of transducer elements in the plurality of transducer elements having a first transducer element and a second transducer element, a spacing between the first transducer element and the second transducer element being based on an intercostal distance.

2. The medical device recited in claim 1, wherein the transducer assembly is connected to the medical device, the medical device further comprising an analysis component configured to evaluate the return signal to determine a variance between the return signal and a transmitted sonic pulse.

3. The medical device recited in claim 2, wherein the analysis component is further configured to determine if variance between the return signal and the transmitted sonic pulse is a product of the Doppler effect.

4. The medical device recited in claim 1, wherein the medical device comprises a portable defibrillator.

5. The medical device recited in claim 1, wherein a second pair of transducer elements in the plurality of transducer elements have a second spacing that is different than the spacing between the first transducer element and the second transducer element.

6. A method for treatment of a cardiac incident, comprising:
transmitting a signal from a transducer, the transducer being oriented so that the signal is transmitted substantially in the direction of a patient's heart, the transmitted signal having a first signal characteristic;
receiving a reflected signal, the reflected signal being at least a partial reflection of the transmitted signal, the reflected signal having a second signal characteristic;
performing an analysis of the reflected signal to determine if the second signal characteristic indicates that the patient's heart is moving, the analysis being based on the Doppler effect.

7. The method recited in claim 6, wherein the analysis compares the second signal characteristic to the first signal characteristic to identify a difference between the first and second signal characteristics.

8. The method recited in claim 7, wherein the first signal characteristic comprises a frequency of the transmitted signal, and further wherein the second signal characteristic comprises a frequency of the reflected signal.

9. The method recited in claim 7, wherein the first signal characteristic comprises a phase of the transmitted signal, and further wherein the second signal characteristic comprises a phase of the reflected signal.

10. The method recited in claim 6, wherein the transmitted signal comprises an ultrasound signal.

11. The method recited in claim 6, further comprising providing a human perceptible indication that the patient's heart is moving.

12. The method recited in claim 11, wherein the human perceptible indication is a notice that a treatment on the patient should be terminated.

13. A medical device, comprising:
a defibrillator port having connectors to deliver a defibrillation shock;
a transponder port having at least one connector to receive a signal indicative of a heart rhythm; and
a processor having an associated memory, the processor being configured to execute operations that include to:
transmit a signal to a transducer through the transducer port, the transducer being oriented so that the signal is transmitted substantially in the direction of a patient's heart, the transmitted signal having a first signal characteristic;
receive a reflected signal, the reflected signal being at least a partial reflection of the transmitted signal, the reflected signal having a second signal characteristic; and
perform an analysis of the reflected signal to determine if the second is signal characteristic indicates that the patient's heart is moving, the analysis being based on the Doppler effect.

14. The medical device recited in claim 13, wherein the analysis compares the second signal characteristic to the first signal characteristic to identify a difference between the first and second signal characteristics.

15. The medical device recited in claim 14, wherein the first signal characteristic comprises a frequency of the transmitted signal, and further wherein the second signal characteristic comprises a frequency of the reflected signal.

16. The medical device recited in claim 14, wherein the first signal characteristic comprises a phase of the transmitted signal, and further wherein the second signal characteristic comprises a phase of the reflected signal.

17. The method recited in claim 13, wherein the transmitted signal comprises an ultrasound signal.

18. The method recited in claim 13, further comprising providing a human perceptible indication that the patient's heart is moving.

19. The method recited in claim 18, wherein the human perceptible indication is a notice that a treatment on the patient should be terminated.
